(19) United States
(12) Patent Application Publication
(10) Pub. No.: US 2025/0248985 A1
(43) Pub. Date: Aug. 7, 2025

(54) TREATMENT OF PARKINSON'S DISEASE AND PARKINSON'S DISEASE PSYCHOSIS

(71) Applicant: VANDA PHARMACEUTICALS INC., Washington, DC (US)

(72) Inventor: Mihael POLYMEROPOULOS, Washington, DC (US)

(21) Appl. No.: 18/856,035

(22) PCT Filed: Apr. 6, 2023
(86) PCT No.: PCT/US2023/065420

§ 371 Date: Oct. 10, 2024

Related U.S. Application Data
(60) Provisional application No. 63/362,959, filed on Apr. 13, 2022.

(51) Int. Cl. A61K 31/454; A61P 25/16

(52) U.S. Cl. CPC A61K 31/454; A61P 25/16

(57) ABSTRACT

The invention relates generally to the treatment of Parkinson's Disease (PD), including Parkinson's Disease psychosis (PDP) with iloperidone.

### CROSS-REFERENCE TO RELATED APPLICATIONS

This application claims the benefit of co-pending U.S. Provisional Patent Application Ser. No. 63/362,959, filed 13 Apr. 2022, which is hereby incorporated herein as though fully set forth.

### BACKGROUND

### Parkinson's Disease

Parkinson's Disease (PD) is a neurodegenerative disease of the central nervous system affecting primarily the motor system. Common motor symptoms include tremors, rigidity, slowness of movement, and difficulty walking. Symptoms are progressive and, as the disease worsens, non-motor symptoms become common. These include cognitive conditions such as depression, anxiety, apathy, and dementia. The cause of PD is unknown and may involve both heritable and environmental factors.

Parkinson's Disease psychosis (PDP) is another common non-motor symptom of PD and may include visual and non-visual hallucinations and delusions. Between 20% and 40% of PD patients report experiencing hallucinations or delusions, with these symptoms being more common in more advanced cases of the disease. Hallucinations have been reported as the strongest predictor for eventual institutionalization of PD patients.

Treatment for PDP include the reduction of dopaminergic therapies, although this alone is often not sufficient to alleviate hallucinations. Antipsychotic therapies have also been employed, including clozapine, quetiapine, and pimavanserin. Clozapine has been shown to be effective in improving psychosis, but is associated with significant side effects, such as agranulocytosis, mortality, seizure, cardiovascular problems, and respiratory problems, all of which can be particularly dangerous in elderly PD patients. Quetiapine has fewer known side effects, but its efficacy has been limited or inconclusive. Pimavanserin is the only approved treatment for PDP but is associated with an increased risk of death in elderly patients with dementia-related psychosis.

### Iloperidone

Iloperidone (1-[4-[3-[4-(6-flouro-1,2-benzisoxazol-3-yl)-1-piperidinyl]propoxy]-3-methoxyphenyl]ethanone) is an atypical antipsychotic disclosed in US Patent RE39,198. It is currently approved by the FDA for the treatment of schizophrenia in adults and sold under the commercial name FANAPT®.

Metabolites of iloperidone, e.g., P88 (also referred to as P-88-8891 or 1-[4-[3-[4-(6-fluoro-1,2-benzisoxazol-3-yl)-1-piperidinyl]propoxy]-3-methoxyphenyl]ethanol), are also useful in the present invention. See, e.g., International Patent Application Publication No. WO03020707, which is incorporated herein by reference. Other iloperidone metabolites include: 1-[4-[3-[4-(6-fluoro-1,2-benzisoxazol-3-yl)-1-piperidinyl]propoxy]-3-hydroxyphenyl]ethanone; 1-[4-[3-[4-(6-fluoro-1,2-benzisoxazol-3-yl)-1-piperidinyl]propoxy]-3-methoxyphenyl]-2-hydroxyethanone; 4-[3-[4-(6-fluoro-1,2-benzisoxazol-3-yl)-1-piperidinyl]propoxy]-3-hydroxy-α-methylbenzene methanol; 4-[3-[4-(6-fluoro-1,2-benzisoxazol-3-yl)-1-piperidinyl]propoxyl-2-hydroxy-5-methoxy-α-methylbenzenemethanol; 1-[4-[3-[4-(6-fluoro-1,2-benzisoxazol-3-yl)-1-piperidinyl]propoxy]-2-hydroxy-5-methoxyphenyl]ethanone; and 1-[4-[3-[4-(6-fluoro-1,2-benzisoxazol-3-yl)-1-piperidinyl]propoxy]-2,5-dihydroxyphenyl]ethanone. See U.S. Pat. No. 5,364,866, and International Patent Application Publication Nos. WO9309276 and WO9511680.

Previous studies have investigated associations between iloperidone efficacy and polymorphisms in genes and gene regions including CFTR, NPAS3, XKR4, TNR, GRIA4, GFRA2, and NUDT9P1. These associations are described in, e.g., U.S. Pat. Nos. 9,328,387, 9,458,507, and 9,080,214. Additionally, associations between CYP2D6 and KCNQ1 genotypes and changes in QT interval following the administration of iloperidone are described in U.S. Pat. Nos. 8,586,610, 9,138,432, 8,999,638, and 9,157,121. Such findings relating to the efficacy of iloperidone aid in selection of the most optimal drug and dosage regimen for a particular patient. This in turn aids in safe and effective treatment of psychotic symptoms, diseases, and disorders, with less trial and error.

### SUMMARY

In one embodiment, the invention provides a method of treating a patient suffering from Parkinson's Disease (PD), the method comprising: administering to said patient iloperidone at a dose effective to alleviate one or more symptom of PD.

In another embodiment, the invention provides, in a method of administering iloperidone to a patient, an improvement comprising: selecting as said patient an individual diagnosed with Parkinson's Disease (PD).

### DETAILED DESCRIPTION

In a study of the efficacy of iloperidone in the treatment of PDP, PD patients 65 years old or older with a clinical diagnosis of PD for at least one year and psychotic symptoms in each week of the prior month are selected for treatment. Psychotic symptoms include visual and/or auditory hallucinations and delusions, including drug-induced delusions.

Patients with a history of significant psychotic disorders prior to or concomitantly with the diagnosis of PD are not included in the study. This includes but is not limited to patients with a history of or concomitant schizophrenia or bipolar disorder. Similarly, patients with evidence of serious or unstable cardiovascular, respiratory, gastrointestinal, renal, hematologic, or other medical disorder are not included in the study. This includes patients with cancer or malignancies.

Iloperidone is administered in an oral tablet form. However, one skilled in the art will recognize that other forms and routes of administration may be employed. Similarly, pharmaceutically-acceptable salts of iloperidone, metabolites of iloperidone, or pharmaceutically-acceptable salts of metabolites of iloperidone may similarly be administered.

In some cases, dosing is titrated to 8 mg/day according to a scheme comprising: 2 mg on day 1, 3 mg on day 2, 4 mg on day 3, 5 mg on day 4, 6 mg on day 5, 7 mg on day 6, and 8 mg on day 7.

This scheme may include: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7.

This scheme may also include: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7, and 8 mg (8 mg in PM) on day 8.

In some cases, dosing is titrated to at least 8 mg/day according to a scheme comprising: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7, and 8 mg (8 mg in PM) on day 8.

This may further include 8 mg (8 mg in PM) on day 9 and thereafter and/or 12 mg (6 mg in AM and 6 mg in PM) on a day after day 8. In other cases, this may further include reducing the dose to 4 mg (2 mg in AM and 2 mg in PM) on a day after day 8.

The efficacy of iloperidone in treating PDP patients is measured by a reduction in the Schedule for the Assessment of Positive Symptoms—Parkinson's Disease (SAPS—PD) scale. Other measures of efficacy may be employed, however, as will be apparent to one skilled in the art.

Study participants are monitored to assess the tolerability, safety, and pharmacokinetics of iloperidone. This includes an assessment of treatment-emergent adverse events (TEAEs), serious adverse events (SAEs), clinically notable abnormal vital signs, electrocardiograms (ECGs), and laboratory analysis of collected specimens.

The foregoing description of various aspects of the invention has been presented for purposes of illustration and description. It is not intended to be exhaustive or to limit the invention to the precise form disclosed, and modifications and variations are possible. Such modifications and variations that may be apparent to a person skilled in the art are intended to be included within the scope of the invention as defined by the accompanying claims.

1. A method of treating a patient suffering from Parkinson's Disease (PD), the method comprising:
administering to said patient iloperidone at a dose effective to alleviate one or more symptom of PD.

2. The method of claim 1, wherein said patient suffers from Parkinson's Disease Psychosis (PDP).

3. The method of claim 2, wherein the one or more symptom of PD is selected from a group consisting of: visual hallucinations, auditory hallucinations, and drug-induced delusions.

4-6. (canceled)

7. The method of claim 1, wherein the dose of iloperidone is 8 mg/day.

8. The method of claim 7, wherein the dose of iloperidone is titrated to 8 mg/day according to the following titration scheme: 2 mg on day 1, 3 mg on day 2, 4 mg on day 3, 5 mg on day 4, 6 mg on day 5, 7 mg on day 6, and 8 mg on day 7.

9. The method of claim 8, wherein the dose of iloperidone is titrated to 8 mg/day according to the following titration scheme: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7.

10. The method of claim 8, wherein the dose of iloperidone is titrated to 8 mg/day according to the following titration scheme: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7, and 8 mg (8 mg in PM) on day 8.

11. The method of claim 1, wherein the dose of iloperidone is at least 8 mg/day.

12. The method of claim 11, wherein the dose of iloperidone is titrated according to the following titration scheme: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7, and 8 mg (8 mg in PM) on day 8.

13. The method of claim 12, wherein the titration scheme further includes: 8 mg (8 mg in PM) on day 9 and thereafter.

14. The method of claim 12, wherein the titration scheme further includes: 12 mg (6 mg in AM and 6 mg in PM) on a day after day 8.

15. The method of claim 12, wherein the titration scheme further includes: 4 mg (2 mg in AM and 2 mg in PM) on a day after day 8.

16. In a method of administering iloperidone to a patient, the improvement comprising:
selecting as said patient an individual diagnosed with Parkinson's Disease (PD).

17. The improvement of claim 16, wherein said patient suffers from Parkinson's Disease Psychosis (PDP).

18. The improvement of claim 17, wherein said patient suffers from at least one symptom selected from a group consisting of: visual hallucinations, auditory hallucinations, and drug-induced delusions.

19-21. (canceled)

22. The improvement of claim 16, wherein the iloperidone is administered to the patient at a dose effective to alleviate one or more symptom of PD.

23. The improvement of claim 22, wherein the dose of iloperidone is 8 mg/day.

24. The improvement of claim 23, wherein the dose of iloperidone is titrated to 8 mg/day according to the following titration scheme: 2 mg on day 1, 3 mg on day 2, 4 mg on day 3, 5 mg on day 4, 6 mg on day 5, 7 mg on day 6, and 8 mg on day 7.

25. The improvement of claim 24, wherein the dose of iloperidone is titrated to 8 mg/day according to the following titration scheme: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7.

26. The improvement of claim 24, wherein the dose of iloperidone is titrated to 8 mg/day according to the following titration scheme: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7, and 8 mg (8 mg in PM) on day 8.

27. The improvement of claim 22, wherein the dose of iloperidone is at least 8 mg/day.

28. The improvement of claim 27, wherein the dose of iloperidone is titrated according to the following titration scheme: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7, and 8 mg (8 mg in PM) on day 8.

29. The improvement of claim 28, wherein the titration scheme further includes: 8 mg (8 mg in PM) on day 9 and thereafter.

30. The improvement of claim 28, wherein the titration scheme further includes: 12 mg (6 mg in AM and 6 mg in PM) on a day after day 8.

31. The improvement of claim 28, wherein the titration scheme further includes: 4 mg (2 mg in AM and 2 mg in PM) on a day after day 8.

32. In a method of administering iloperidone to a patient, the improvement comprising:
administering the iloperidone to said patient according to the following titration scheme: 2 mg on day 1, 3 mg on day 2, 4 mg on day 3, 5 mg on day 4, 6 mg on day 5, 7 mg on day 6, and 8 mg on day 7.

33. The improvement of claim 32, wherein the titration scheme includes: 2 mg (1 mg in AM and 1 mg in PM) on day 1, 3 mg (1 mg in AM and 2 mg in PM) on day 2, 4 mg (2 mg in AM and 2 mg in PM) on day 3, 5 mg (2 mg in AM and 3 mg in PM) on day 4, 6 mg (3 mg in AM and 3 mg in PM) on day 5, 7 mg (3 mg in AM and 4 mg in PM) on day 6, 8 mg (4 mg in AM and 4 mg in PM) on day 7.

34. The improvement of claim 33, wherein the titration scheme further includes: 8 mg (8 mg in PM) on day 8.

35. The improvement of claim 34, wherein the titration scheme further includes: 12 mg (6 mg in AM and 6 mg in PM) on a day after day 8.

36. The improvement of claim 34, wherein the titration scheme further includes: 4 mg (2 mg in AM and 2 mg in PM) on a day after day 8.
